(19) United States
(12) Patent Application Publication
(10) Pub. No.: US 2014/0256639 A1
(43) Pub. Date: Sep. 11, 2014

(54) PEPTOID NEUTRALIZING AGENTS

(71) Applicant: The Regents of the University of California, Oakland, CA (US)

(72) Inventors: Dallas L. Rabenstein, Riverside, CA (US); Mark H. Hamza, Riverside, CA (US); Bruce K. Ford, Riverside, CA (US)

(21) Appl. No.: 14/180,877

(22) Filed: Feb. 14, 2014

Related U.S. Application Data
(60) Provisional application No. 61/764,946, filed on Feb. 14, 2013.

(51) Int. Cl. C07K 7/08; C07K 7/06; C07K 5/08

(52) U.S. Cl. CPC C07K 7/08; C07K 5/08; C07K 7/06
USPC 514/13.7; 530/331; 530/329; 530/328; 530/327; 514/21.9; 514/21.8; 514/21.6; 514/21.5

(57) ABSTRACT

The disclosure provides for one or more peptoids, methods of manufacture thereof, and methods of use thereof, including the use of one more peptoids in neutralizing the anticoagulant activity of heparin.

### CROSS REFERENCE TO RELATED APPLICATIONS

This application claims priority under 35 U.S.C. §119 from Provisional Application Ser. No. 61/764,946, filed Feb. 14, 2013, the disclosure of which is incorporated herein by reference.

### TECHNICAL FIELD

The disclosure provides for peptoids and methods of use thereof, including neutralizing the anticoagulant activity of heparin.

### BACKGROUND

Heparin, a highly-sulfated glycosaminoglycan, is used as a blood anticoagulant in medicine, veterinary medicine and biological research. Protamine is used to counteract the anticoagulant activity of heparin.

### SUMMARY

The disclosure provides for one or more peptoids having varying lengths and side chains that can neutralize the anticoagulant activity of heparin. In a particular embodiment, one or more peptoids of the disclosure can be used to mimic the effects of Protamine. In a further embodiment, one or more peptoids of disclosure can be used to neutralize or counteract the anticoagulant activity of heparin.

The disclosure provides a peptoid comprising the structure of Formula I(a):

wherein, Z is an integer from 1 to 10; R¹-R³ are each individually selected from optionally substituted (C₁-C₁₂) alkyl, optionally substituted (C₁-C₁₂) alkenyl, optionally substituted (C₁-C₁₂) alkynyl, optionally substituted aryl, optionally substituted cycloalkyl, optionally substituted cycloalkenyl, and optionally substituted heterocycle; wherein R³ is chiral so that the defined group is either the R-isomer or the S-isomer, R² is an unsubstituted hydrocarbon or hydrophobic group, and R¹ either comprises a cationic group and/or is optionally substituted with a cationic group. In one embodiment, the cationic group is selected from the group consisting of ammonium and guanidinium. In another further embodiment, R¹ is selected from the group consisting of:

In yet another embodiment, the chiral group is either (S)-1-phenylethylamine or (R)-1-phenylethylamine. In yet another embodiment, the unsubstituted hydrocarbon or hydrophobic group is selected from the group consisting of a straight unsubstituted (C₁-C₉) hydrocarbon group, a branched unsubstituted (C₁-C₉) hydrocarbon group, an unsubstituted (C₁-C₉) allylic group, and an unsubstituted (C₁-C₉) benzylic group. In yet another embodiment, the unsubstituted hydrocarbon or hydrophobic group is selected from the group consisting of:

In another embodiment, Z is either 3 or 4. In another embodiment, the peptoid forms a secondary structure similar to polyproline type I or polyproline type II peptide helices. In a further embodiment, the peptoid forms a secondary structure similar to a polyproline type I helix where all of the R groups comprising cationic groups project from one face of the helix. In one embodiment, the peptoide comprises the structure of Formula I(a):

wherein, Z is an integer from 1 to 10; R¹-R² are each individually

and wherein R³ is (S)-1-phenylethylamine or (R)-1-phenylethylamine. In one embodiment, the peptoid has the structure:

The disclosure also provide a pharmaceutical composition comprising a pharmaceutically acceptable carrier and a peptoid of the disclosure.

The disclosure also provides a method of neutralizing the anticoagulant activity of heparin comprising administering a peptide or pharmaceutical preparation thereof to a subject in need of reduced anticoagulant activity. In another embodiment, the peptide is administered parenterally.

The disclosure also provide a method of making a peptoid of the disclosure comprising: (A) coupling a resin bound amine or monomer to bromoacetic acid using a peptide coupling agent to form an attached submonomer portion; (B) adding a second amine to the submonomer portion using an SN² reaction mechanism so as to form a full monomer, wherein an amine nucleophile forms a bond with an electrophilic alkyl bromide carbon such that the bromine is displaced as a leaving group, and repeating (A) and (B) until the peptoid is synthesized. In one embodiment, the peptide coupling agent is N,N′-diisopropylcarbodiimide. In another embodiment, a cleavage solution is added after the synthesis of the peptoid to cleave the peptoid from the resin and to remove amine protecting groups. In yet another embodiment, the peptoid is purified by using prepatory reverse phase HPLC. In another embodiment, the method utilizes a peptide synthesizer which may or may not be further connected to a computer.

### DESCRIPTION OF DRAWINGS

FIG. 1A-B: (A) shows a major repeating L-idurouic-(1-4)-D-glucosamine disaccharide of heparin. (B) presents the structure of Heparin. Heparin has a high negative charge density due to its large number of anionic functional groups.

FIG. 2A-B presents the general structure of (A) a tetrapeptoid and (B) a tetra peptide. For peptoids, the R group side chain is connected directly to the back bone amide nitrogen. By contrast, the R group side chain is connected to the alpha carbon in peptides.

FIG. 3 provides an embodiment of the disclosure of a submonomer approach to synthesize peptoids of the disclosure using resin. The complete monomers are produced on resin in two steps, each step comprising a submonomer.

FIG. 4 presents a peptoid of the disclosure. The peptoid is an s9mer and corresponds to sequence H—[N(Orn)-N(Bu)—N(Spe)]₃—NH₂.

FIG. 5 provides representative cis and trans conformations of peptoid backbones. The conformations affect the secondary structures of peptoids disclosed herein.

FIG. 6A-E presents embodiments of the disclosure of peptoids with various side chain substitutions. The peptoids, as shown, are designated (A) s9merBz:(H—[N(Orn)-N(Bz)-N(Spe)]₃—NH₂); (B) s12merG:(H—[N(Arg)-N(Bu)-N(Spe)]₄—NH₂); (C) s9merCadG (H—[N(CadG)-N(Bu)-N(Spe)]₃—NH₂); (D) s9merMe: (H-[N(Orn)-N(Me)-N(Spe)]₃—NH₂; and (E) s12merArgArg H—[N(Arg)-N(Arg)-N(Spe)]₄-NH₂.

FIG. 7 provides a helical model of peptoids of the disclosure. The model predicts that the peptoids disclosed herein, by forming a helical structure, can result in the alignment of the cationic sites on one face of the peptoid. The model further predicts that the distances between the cationic sites to be around 6.1 Å. This distance is similar to the distance between anionic sites of heparin.

FIG. 8 presents the circular dichroism (“CD”) spectra for two peptoids of the disclosure. S12mer and R12mer have identical sequences but are S and R isomers. The S12-mer peptoid is shown as the bottom line, and the R12-mer is shown as the top line. The two curves produced by the CD spectra mirror each other and are indicative of a right and left-handed helix respectively.

FIG. 9 presents a graph demonstrating a correlation between the lengths of peptoids (e.g., 3 to 9 peptoids long (h-[n(orn)-n(bu)-n(spg)], wherein n=1-4)) of the disclosure with heparin retention times, using Heparin Affinity Chromatography (“HAC”).

FIG. 10 presents a calibration curve obtained from the natural log of the isothermal titration calorimetry (“ITC”) binding constants and the heparin retention times vs. overall peptoid length from 3 to 12 monomers in length for peptoids with ammonium and guanidinium side chains using HAC.

FIG. 11A-B presents an ITC plot for the endothermic reaction of (A) a 9-mer and (B) S-12mer peptoids of the disclosure with heparin.

FIG. 12A-B presents an integrated ITC curve for a reaction between 0.20 mM of (A) a 9mer peptoid of the disclosure and (B) a 12mer peptoid, in a sample cell and 0.25 mM heparin in a titration syringe.

FIG. 13 presents a bar graph of estimated heparin binding constants determined by ITC for ammonium-bearing peptoids of the disclosure and binding constants estimated for guanidinium-bearing peptoids of the disclosure.

FIG. 14A-C presents the reaction schemes involved in the Coatest® assay used with the peptoids of the disclosure. In (A), an excess of AT is added to a specific concentration of heparin forming a complex between Hep and AT leaving an excess of AT. In (B) an excess of FXa is added which combines with the Hep-AT complex to form Hep-AT-FXa leaving an excess of FXa which mediates the following reaction shown in (C). In (C), the remaining amount of FXa hydrolyses the chromogenic substrate S-2222 thus liberating the chromophoric group, pNA. The color (yellow) is then read photometrically at 405 nm.

FIG. 15 provides a plot of the results from a Coatest assay of six representative peptoids of the disclosure in comparison to protamine sulfate. The assay measures percent recovery of FXa required to restore heparin activity. As the concentration of peptoid of the disclosure is increased, the heparin is bound to the peptoid instead of the heparin-AT-FXa complex resulting in an increase in free FXa, which catalyzes the cleavage of the chromogenic substrate S-2222 shown in the reaction scheme given above. The vertical axis is percent FXa recovery and the horizontal axis is the concentration of peptoid or protamine sulfate in micrograms per millimeter.

FIG. 16A-B shows peptoid structures for (A) ammonium bearing peptoids and (B) guanidinium bearing peptoids.

FIG. 17A-B show the HAC retention times of peptoid number 4-20 in Table 2.

### DETAILED DESCRIPTION

As used herein and in the appended claims, the singular forms “a,” “and,” and “the” include plural referents unless the context clearly dictates otherwise. Thus, for example, reference to “a peptoid” includes a plurality of such peptoids and reference to “cationic groups” includes reference to one or more cationic groups and equivalents thereof known to those skilled in the art, and so forth.

Unless defined otherwise, all technical and scientific terms used herein have the same meaning as commonly understood to one of ordinary skill in the art to which this disclosure belongs. Although any methods and reagents similar or equivalent to those described herein can be used in the practice of the disclosed methods and compositions, the exemplary methods and materials are now described.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

All publications mentioned herein are incorporated herein by reference in full for the purpose of describing and disclosing the methodologies, which are described in the publications, which might be used in connection with the description herein. The publications discussed above and throughout the text are provided solely for their disclosure prior to the filing date of the present application. Nothing herein is to be construed as an admission that the inventors are not entitled to antedate such disclosure by virtue of prior disclosure. Moreover, with respect to any term that is presented in one or more publications that is similar to, or identical with, a term that has been expressly defined in this disclosure, the definition of the term as expressly provided in this disclosure will control in all respects.

The terms “treat”, “treating” and “treatment”, as used herein, refer to ameliorating symptoms associated with a condition, for example, heparin overdose, including preventing or delaying the onset of symptoms, and/or lessening the severity or frequency of symptoms of the condition.

The terms “subject” and “individual” are defined herein to include animals, such as mammals, including but not limited to, primates, cows, sheep, goats, horses, dogs, cats, rabbits, guinea pigs, rats, mice or other bovine, ovine, equine, canine, feline, rodent, or murine species. In a particular embodiment, the mammal is a human.

Heparin is a naturally-occurring anticoagulant produced in the human body by mast cells and basophils. The most common source of commercially available heparin is from porcine intestine and bovine lung. Heparin has long been known to possess anticoagulant properties that have made it especially useful in medical settings that require the inhibition of blood clot formation such as during major surgery where blood is circulated extracorporeally in heart-lung machines or during kidney dialysis, where the surfaces of all polymer lines and other equipment that may come into direct contact with blood is functionalized by binding the surface to chains of heparin thus producing a buffered environment that protects the blood from clotting through the intrinsic coagulation pathway (Cox, M., Nelson, D. Principles of Biochemistry; Lehninger, 2004). In addition to its anticoagulant properties, heparin can also mediate the release of hepatic and lipoprotein lipases, regulate angiogenesis and growth of tumors, modulate inflammation, and may also have antiviral properties.

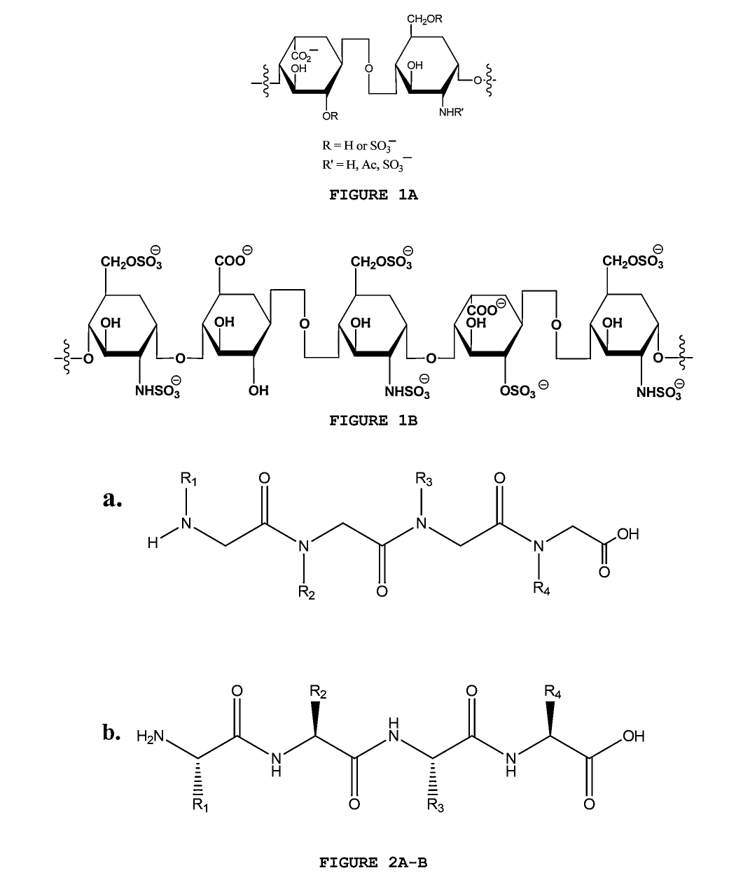

Structurally, heparin consists of linear repeating units of variably sulfated disaccharides (see e.g., FIG. 1) with molecular weight that varies from 3-50 kD, but most commercial sources range from 12-15 kD (Linhardt et al., R. Chem. Indust. 1991, 2, 45-50). The partially sulfated ester and amide residues under physiological pH conditions exist in the deprotonated form resulting in a net negative charge density that is one of the greatest in any known biological molecule (Rabenstein, Nat. Prod. Rep. 2002, 19, 312-331).

In some clinical situations, it is necessary to neutralize the anticoagulant activity of heparin. For example, bleeding complications are a side effect in some 10-150 of patients. To neutralize the anticoagulant activity of heparin, protamine, a low molecular weight (4.5 kD) arginine rich protein, is used. The arginine residues in protamine are cationic and can therefore bind with the anionic sulfates of heparin.

Intravenous injections of protamine, however, may cause life threatening side effects such as bradycardia, hypotension and other anaphylactic symptoms (Gupta et al., J Vasc Surg. 1989, 9(2):342-50). Accordingly, protamine should only be given when resuscitation techniques and treatment of anaphylactoid shock are readily available. Moreover, protamine has been shown to be incompatible with certain antibiotics, including several of the cephalosporins and penicillins. The scientific community has advocated a need for protamine replacement as anaphylactic reactions to protamine sulfate is unpredictable, and protamine is the only drug currently available to reverse heparin anticoagulation (Dayal et al., Drug Intell. Clin. Pharm. 1988, 22(3):209-11; Guyton, A. C., Hall, J. E. Textbook of Medical Physiology, 2006; and Levy et al., Anesthesia & Analgesia 2009, 108(3):692-694).

While a protamine alternative based on heparinase I from Flavobacterium heparinum showed initial promise, it was found to be not as safe as protamine (Stafford-Smith et al., Anesthesiology 2005, 103(2):229-240).

Most of the current research on protamine alternatives has focused on heparin-binding peptides including synthetic peptides that have the sequence of heparin-binding domains of heparin-binding proteins, such as heparin interacting protein (HIP²) identified in human uterine epithelial cells and cell lines (Carr et al., J Cardiovasc Surg (Torino) 1999, 40:659-66). The amino acid sequence of the heparin binding domain of HIP² is CPKAKAKAKAKDQTK (SEQ ID NO:1). This sequence contains one arginine and six lysine residues that under physiological conditions would carry a cationic charge. This cationic charge could be expected to facilitate the interaction of HIP² with the negatively charged heparin.

Additional experiments with modified peptides of a similar sequence have also been shown to bind with high affinity to heparin and neutralize the anticoagulant activity (Liu et al., D. D. Proc. Natl. Acad. Sci. U.S.A. 1997, 94, 1739-1744). Two synthetic peptide analogues comprising the HIP² heparin-binding domain were found to have significant binding affinities to heparin (Rabbenstein, D. A., Wang, J Biochem 45: 15740-747, 2006). The synthetic peptide analogues have the sequence Ac-SRGKAKVKAKVKDQTK-NH₂ (SEQ ID NO:2), and were differentiated by consisting only of L-amino acids (L-HIPAP) or D-amino acids (D-HIPAP). Both peptide analogues were found to be equally effective in neutralizing heparin activity by using the Coatest® heparin in vitro assay. However, D-HIPAP was found to be more stable than L-HIPAP, as L-HIPAP was more susceptible to degradation by proteases under physiological conditions due to being comprised of naturally occurring amino acids. Peptoids are similar to peptides comprised of D-amino acids, as they are likewise completely resistant to proteolysis (Moos et al., Drug Dev. Res. 1995, 15:5282-5335).

Studies with peptoids with alpha chiral side chains showed that these peptoids could form helical structures similar to polyproline type I peptide helices (Barron et al., J. Am. Chem. Soc. 2003, 125(44):13525-13530). Moreover, the secondary structures of these peptoids do not involve hydrogen bonding and are therefore are resistant to denaturing by solvents, temperature, or by chemical denaturants (Barron et al., Biopolymers 2002, 63:12-20). Furthermore, as functionalizing the alpha nitrogen is facile, an incredibly diverse population of peptoids can be generated (Wetzler et al., Biopolym. Pept. Sci. 2011, 96(5):556-560). Peptoids also have enhanced membrane permeability and better immunogenicity profiles in comparison to naturally occurring peptides or proteins, such as Protamine.

A major design consideration in constructing peptoids of the disclosure were that the peptoids comprise multiple cationic side chains spaced so that these side chains could interact with heparin anionic groups through electrostatic interactions. By performing peptoid structure modeling, it was found that by adding monomers with cationic side chains at the third position of every third monomer so as to generate a repeating trimer sequence would result in a peptoid that had the potential to electrostatically bind heparin if the resulting peptoid could form a helix. Further modeling, indicated that secondary helical structures could be produced by including alpha chiral side chains at the first position of every third monomer so that the resulting helices would have pitch of 3 residues per turn. Cationic side chains, such as ammonium and guanidinium, were predicted to interact with the many deprotonated sulfate esters, sulfamates, and carboxylates of heparin.

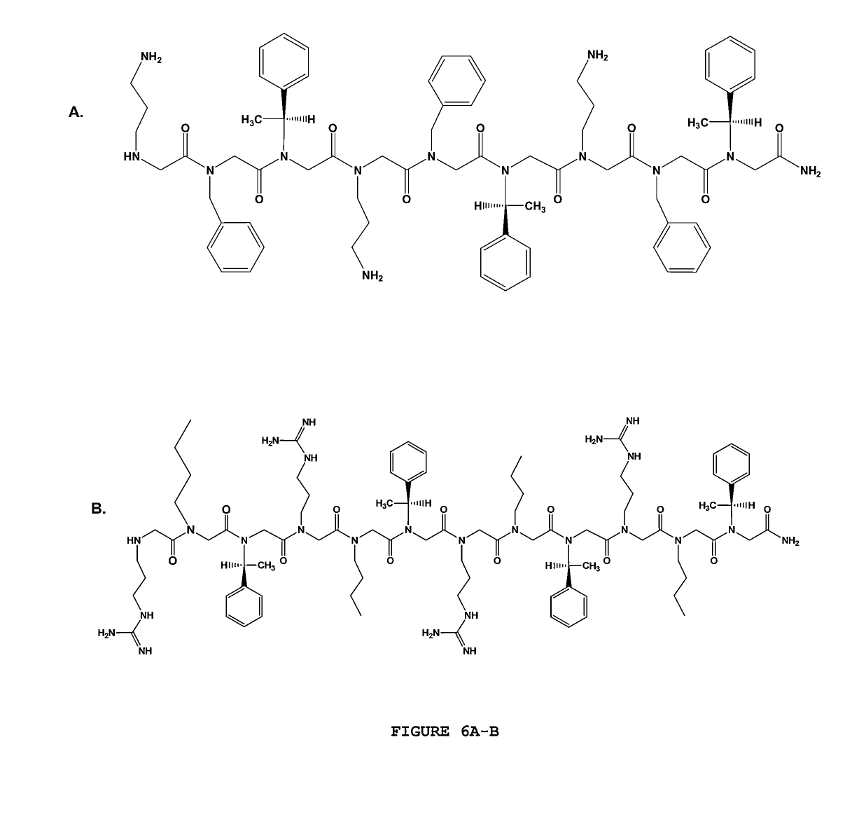

For peptides, the peptide bond has a double bond character and therefore when peptides are folded they are either exclusively in the cis or in the trans isomer. By contrast, for peptoids, cis/trans isomerism is so common that in a given peptoid the true secondary structure will be a mixture of cis and trans isomers (e.g., see FIG. 5). An instance where the entire peptoid will consist of the same secondary structure, such as a poly proline I or II helix, is if the entire sequence is made of alpha chiral side chains, such as polySpe15, a peptoid of the disclosure.

Despite any deviation away from the idealized structure envisioned in the original rational design, the disclosure provides for one or more peptoids with multiple cationic side chains extending away from the peptoid backbone that bind with great affinity to the anionic deprotonated esters and amides of heparin as seen in the isothermal titration calorimetry (“ITC”) and heparin affinity chromatography (“HAC”) experiments. Additionally, the results of the Coatest® Heparin Assay demonstrate that the peptoids of the disclosure electrostatically bind to heparin despite peptoids disclosed herein having structural variations.

It was found through experimentation that certain alterations to the interacting side chains, such as increasing side chain length, were instrumental in enhancing the binding affinities of one or more peptoids disclosed herein to heparin. The lengths of peptoids of the disclosure were also varied to consist from 3 to 12 monomers, so as to examine the effect of peptoid length to heparin binding affinity. As expected, there was a direct correlation between overall peptoid length, and thus the number of positive sites on the peptoid, and heparin binding affinity. It was also found that peptoid length affected the solubility of the peptoids in aqueous media. Of the tested peptoids, peptoids consisting of nine monomers had the best solubility in aqueous media.

In a particular embodiment, one or more peptoids of disclosure are between 1 to 30 monomers in length, 2 to 20 monomers in length, 3 to 15 monomers in length, 3 to 12 monomers in length, 7 to 12 monomers in length, or 5 to 10 monomers in length.

The disclosure further provides for peptoids disclosed herein built using a general scaffold comprising the following sequence: H—[N(Cationic side chain)-N(alkyl or benzyl side chain)-N(spe)]₃—NH₂, where spe is (S)-1-phenylethylamine. The cationic side chain comprises a cationic group, such as an ammonium or a guanidinium group, wherein the cationic group is attached to carbon chain as defined herein.

A generalized structure of a (A) peptoid of the disclosure in comparison to (B) a peptide is presented in FIG. 2A-B. As can also be seen in FIG. 2A-B, unlike natural peptides, the side chain is connected directly to the back bone amine nitrogen of the peptoid as opposed to the alpha carbon as is the case with peptides.

In one embodiment, the disclosure provides for one or more peptoids comprising Formula I:

wherein,

Z is an integer from 1 to 10 (e.g., 1, 2, 3, 4, 5, 6, 7, 8, 9, or 10);

A¹-A⁶ are each individually selected from the group comprising H, D, optionally substituted (C₁-C₆) alkyl, optionally substituted (C₁-C₆) alkenyl, optionally substituted (C₁-C₆) alkynyl, optionally substituted aryl, optionally substituted cylcoalkyl, optionally substituted cycloalkyenyl, optionally substituted heterocycle, hydroxyl, ester, ether, carbonyl, amine, cyano, nitro, thiol, sulfide, and sulfoxide;

X¹ and X² are each individually selected from the group comprising H, D, optionally substituted (C₁-C₉) alkyl, optionally substituted (C₁-C₉) alkenyl, optionally substituted (C₁-C₉) alkynyl, optionally substituted aryl, optionally substituted cycloalkyl, optionally substituted cycloalkyenyl, optionally substituted heterocycle, hydroxyl, ester, ether, carbonyl, amine, cyano, nitro, thiol, sulfide, and sulfoxide;

R¹-R³ are each individually selected from optionally substituted (C₁-C₁₂) alkyl, optionally substituted (C₁-C₁₂) alkenyl, optionally substituted (C₁-C₁₂) alkynyl, optionally substituted aryl, optionally substituted benzyl, optionally substituted cycloalkyl, optionally substituted cycloalkyenyl, and optionally substituted heterocycle;

wherein one of R¹-R³ is chiral so that the defined group is either the R-isomer or the S-isomer, one of R¹-R³ can be an unsubstituted hydrocarbon or hydrophobic group, and one or more of R¹-R³ either comprises a cationic group or is optionally substituted with a cationic group.

In a further embodiment, the disclosure provides for one or more peptoids comprising Formula I(a):

wherein,

Z is an integer from 1 to 5 (e.g., 1, 2, 3, 4, 5);

R¹-R³ are each individually selected from optionally substituted (C₁-C₁₂) alkyl, optionally substituted (C₁-C₁₂) alkenyl, optionally substituted (C₁-C₁₂) alkynyl, optionally substituted aryl, optionally substituted benzyl, optionally substituted cycloalkyl, optionally substituted cycloalkyenyl, and optionally substituted heterocycle;

wherein R³ is chiral so that the defined group is either the R-isomer or the S-isomer, R² is can be unsubstituted hydrocarbon or hydrophobic group, and R¹ and optionally R² can either comprise a cationic group or is optionally substituted with a cationic group. Examples of cationic groups include, but are not limited to, ammonium and guanidinium.

The disclosure provides for one or more peptoids disclosed herein that can form a helix like secondary structure. One or more peptoids of the disclosure comprise alpha chiral side chains starting with the first residue from the C-terminus and at every third residue (i and i+3). The helicity of peptoids disclosed herein were confirmed by using circular dichroism (“CD”). The general design of the heparin binding peptoids disclosed herein was based upon the repeating trimer sequences described above, which result in a helical structure with all cationic side chains on one face, which would then align with the anionic side chains of the linear heparin polymer. For this disclosure, a list of the side chain designations along with the corresponding side chain structures are presented in Table 1.

```
TABLE 1
Cationic Monomers
  N(Orn)
  N(Lys)
  N(Cad)
  N(Arg)
  N(Lys)G
  N(Cad)G
Alkyl/Hydrophobic Monomers
  N(Me)
  N(Eth)
  N(Pr)
  N(Bu)
  N(All)
  N(Bz)
Chiral Monomers
  N(Spe)
  N(Rpe)
```

The disclosure provides for peptoids that would bind heparin with high affinity. Such peptoids find use as replacements for protamine for the neutralization of the anticoagulant activity of heparin. To this end, a library of peptoids was synthesized and optimized by making alterations to the N-side chains of the monomers in order to generate peptoids with improved heparin affinity. All of the synthesized peptoids were based upon sequences of repeating trimers with the cation-bearing side chains positioned to be on one face of a helical secondary structure.

In a particular embodiment, one or more peptoids disclosed herein comprise two cationic groups on the repeating trimer sequence to emulate the structure of protamine. In a further embodiment, one or more peptoids disclosed herein have between 1 to 10 cationic groups, between 2 to 8 cationic groups, or between 3 to 7 cationic groups.

The peptoids were synthesized using the submonomer approach developed by Zuckermann, which method and teachings are incorporated herein (Zuckermann et al., J. A. C. S. 1992, 114(26):10646-10647). In the first step, a resin bound amine is coupled with DIC(N,N′-diisopropylcarbodiimide) to bromoacetic acid. A second amine is then added to the submonomer portion which is now bound to the resin with an electrophilic bromide leaving group. The nucleophilc amine replaces the leaving group with the production of the full monomer, and the process is continued producing ultimately a full peptoid bound to the resin with reactive side chains attached to the backbone nitrogens, both of which are removed with trifluoroacetic acid (“TFA”) during final cleavage.

Following synthesis and purification by reverse phase high performance liquid chromatography (“HPLC”), peptoids of the disclosure were analyzed by Isothermal Titration calorimetry (“ITC”) to obtain binding constants and thermodynamic parameters for the interaction between peptoids and heparin. Heparin Affinity Chromatography (“HAC”) was also used to study relative binding affinities between heparin and the peptoids synthesized herein. The retention times obtained by this method were compared with the binding data from ITC. Circular Dichroism (“CD”) was conducted on the peptoids disclosed herein to gain insight into their secondary structure in solution. Finally, Coatest® assays were performed with peptoids disclosed herein showing that the peptoids act similarly to protamine by restoring coagulation through the Factor Xa (“FXa”) pathway.

The disclosure provides for a pharmaceutical composition comprising one or more peptoids disclosed herein, or a pharmaceutically acceptable salt, solvate, or prodrug thereof, as an active ingredient, in combination of one or more pharmaceutically acceptable carriers, pharmaceutically acceptable vehicle, carrier, diluent, or excipient, or mixtures thereof.

Disclosed herein are pharmaceutical compositions in modified release dosage forms, which comprise one or more peptoids disclosed herein, or a pharmaceutically acceptable salt, solvate, or prodrug thereof, and one or more release controlling excipients or carriers as described herein. Suitable modified release dosage vehicles include, but are not limited to, hydrophilic or hydrophobic matrix devices, water-soluble separating layer coatings, enteric coatings, osmotic devices, multiparticulate devices, and combinations thereof. The pharmaceutical compositions may also comprise non-release controlling excipients or carriers.

Additionally disclosed are pharmaceutical compositions in a dosage form that has an instant releasing component and at least one delayed releasing component, and is capable of giving a discontinuous release of one or more peptoids of the disclosure in the form of at least two consecutive pulses separated in time from 0.1 up to 24 hours.

The pharmaceutical compositions comprise one or more peptoids disclosed herein, or a pharmaceutically acceptable salt, solvate, or prodrug thereof, and one or more release controlling and non-release controlling excipients or carriers, such as those excipients or carriers suitable for a disruptable semi-permeable membrane and as swellable substances.

Provided herein are pharmaceutical compositions that comprise a concentration of about 0.1 to about 500 mg/mL, about 1 to about 250 mg/mL, about 2 to about 100 mg/mL, about 1 mg/mL, about 2 mg/mL, about 3 mg/mL, about 5 mg/mL, about 10 mg/mL, about 15 mg/mL, about 20 mg/mL, about 30 mg/mL, about 40 mg/mL, about 50 mg/mL, about 100 mg/mL, about 500 mg/mL of one or more peptoids disclosed herein, in the form for parental administration.

The pharmaceutical compositions comprising one or more peptoids disclosed herein may be disclosed in unit-dosage forms or multiple-dosage forms. Unit-dosage forms, as used herein, refer to physically discrete units suitable for administration to human and animal subjects and packaged individually as is known in the art. Each unit-dose contains a predetermined quantity of one or more peptoids disclosed herein which is sufficient to produce the desired therapeutic effect, in association with the required pharmaceutical carriers or excipients. Examples of unit-dosage forms include ampouls, syringes, and individually packaged tablets and capsules. Unit-dosage forms may be administered in fractions or multiples thereof. A multiple-dosage form is a plurality of identical unit-dosage forms packaged in a single container to be administered in segregated unit-dosage form. Examples of multiple-dosage forms include vials, bottles of tablets or capsules, or bottles of pints or gallons.

One or more peptoids disclosed herein may be administered alone, or in combination with one or more other compounds disclosed herein, one or more other active ingredients. The pharmaceutical compositions that comprise one or more peptoids disclosed herein may be formulated in various dosage forms for oral, parenteral, and topical administration. The pharmaceutical compositions comprising one or more peptoids may also be formulated as a modified release dosage form, including delayed-, extended-, prolonged-, sustained-, pulsatile-, controlled-, accelerated- and fast-, targeted-, programmed-release, and gastric retention dosage forms. These dosage forms can be prepared according to conventional methods and techniques known to those skilled in the art (see, Remington: The Science and Practice of Pharmacy, supra; Modified-Release Drug Delivery Technology, Rathbone et al., Eds., Drugs and the Pharmaceutical Science, Marcel Dekker, Inc., New York, N.Y., 2002, Vol. 126).

The pharmaceutical compositions comprising one or more peptoids disclosed herein may be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

The pharmaceutical compositions comprising one or more peptoids disclosed herein may be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, and subcutaneous administration.

The pharmaceutical compositions comprising one or more peptoids disclosed herein may be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, Remington: The Science and Practice of Pharmacy, supra).

The pharmaceutical compositions comprising one or more peptoids of the disclosure intended for parenteral administration may include one or more pharmaceutically acceptable carriers and excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

Suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection. Non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, dimethylacetamide, and dimethylsulfoxide.

Suitable antimicrobial agents or preservatives include, but are not limited to, phenols, cresols, mercurials, benzyl alcohol, chlorobutanol, methyl and propyl p-hydroxybenzates, thimerosal, benzalkonium chloride, benzethonium chloride, methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants are those as described herein, including bisulfite and sodium metabisulfite. Suitable local anesthetics include, but are not limited to, procaine hydrochloride. Suitable suspending and dispersing agents are those as described herein, including sodium carboxymethylcelluose, hydroxypropyl methylcellulose, and polyvinylpyrrolidone. Suitable emulsifying agents include those described herein, including polyoxyethylene sorbitan monolaurate, polyoxyethylene sorbitan monooleate 80, and triethanolamine oleate. Suitable sequestering or chelating agents include, but are not limited to EDTA. Suitable pH adjusting agents include, but are not limited to, sodium hydroxide, hydrochloric acid, citric acid, and lactic acid. Suitable complexing agents include, but are not limited to, cyclodextrins, including α-cyclodextrin, β-cyclodextrin, hydroxypropyl-β-cyclodextrin, sulfobutylether-β-cyclodextrin, and sulfobutylether 7-β-cyclodextrin (CAPTISOL®, CyDex, Lenexa, Kans.).

The pharmaceutical compositions comprising one or more peptoids disclosed herein may be formulated for single or multiple dosage administration. The single dosage formulations are packaged in an ampule, a vial, or a syringe. The multiple dosage parenteral formulations can contain an antimicrobial agent at bacteriostatic or fungistatic concentrations. All parenteral formulations should be sterile, as known and practiced in the art.

In one embodiment, the pharmaceutical compositions comprising one or more peptoids of the disclosure are formulated as ready-to-use sterile solutions. In another embodiment, the pharmaceutical compositions comprising one or more peptoids of the disclosure are formulated as sterile dry soluble products, including lyophilized powders and hypodermic tablets, to be reconstituted with a vehicle prior to use. In yet another embodiment, the pharmaceutical compositions comprising one or more peptoids of the disclosure are formulated as ready-to-use sterile suspensions. In yet another embodiment, the pharmaceutical compositions comprising one or more peptoids of the disclosure are formulated as sterile dry insoluble products to be reconstituted with a vehicle prior to use. In still another embodiment, the pharmaceutical compositions comprising one or more peptoids of the disclosure are formulated as ready-to-use sterile emulsions.

The pharmaceutical compositions comprising one or more peptoids disclosed herein may be formulated as immediate or modified release dosage forms, including delayed-, sustained, pulsed-, controlled, targeted-, and programmed-release forms.

The pharmaceutical compositions comprising one or more peptoids of the disclosure may be formulated as a suspension, solid, semi-solid, or thixotropic liquid, for administration as an implanted depot. In one embodiment, the pharmaceutical compositions comprising one or more peptoids disclosed herein are dispersed in a solid inner matrix, which is surrounded by an outer polymeric membrane that is insoluble in body fluids but allows one or more peptoids in the pharmaceutical compositions diffuse through.

Suitable inner matrixes include polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers, such as hydrogels of esters of acrylic and methacrylic acid, collagen, cross-linked polyvinylalcohol, and cross-linked partially hydrolyzed polyvinyl acetate.

Suitable outer polymeric membranes include polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer.

The disclosure also provides a method of neutralizing the anticoagulant activity of heparin comprising administering one or more peptoids disclosed herein or pharmaceutical compositions comprising one or more peptoids disclosed herein.

Disclosed herein are methods for treating a subject, including a human, in need of neutralizing the anticoagulant activity of administered heparin comprising administering to the subject a therapeutically effective amount of one or more peptoids as disclosed herein, or a pharmaceutically acceptable salt, solvate, or prodrug thereof, so as to neutralize or reverse the anticoagulant activity of heparin.

The dose may be in the form of one, two, three, four, five, six, or more sub-doses that are administered at appropriate intervals per day. The dose or sub-doses can be administered in the form of dosage units containing from about 0.1 to about 1000 milligrams, from about 0.1 to about 500 milligrams, or from 0.5 about to about 100 milligrams active ingredient(s) per dosage unit, and if the condition of the patient requires, the dose can, by way of alternative, be administered as a continuous infusion.

In certain embodiments, an appropriate dosage level is about 0.01 to about 100 mg per kg patient body weight per day (mg/kg per day), about 0.01 to about 50 mg/kg per day, about 0.01 to about 25 mg/kg per day, or about 0.05 to about 10 mg/kg per day, which may be administered in single or multiple doses. A suitable dosage level may be about 0.01 to about 100 mg/kg per day, about 0.05 to about 50 mg/kg per day, or about 0.1 to about 10 mg/kg per day. Within this range the dosage may be about 0.01 to about 0.1, about 0.1 to about 1.0, about 1.0 to about 10, or about 10 to about 50 mg/kg per day.

One or more peptoids disclosed herein may also be combined or used in combination with other agents useful in neutralizing the anticoagulant activity of heparin. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced).

Such other agents, adjuvants, or drugs, may be administered, by a route and in an amount commonly used therefor, simultaneously or sequentially with a compound as disclosed herein. When one or more peptoids disclosed herein is used contemporaneously with one or more other drugs, a pharmaceutical composition containing such other drugs in addition to one or more peptoids of the disclosure may be utilized, but is not required. Accordingly, the pharmaceutical compositions disclosed herein include those that also contain one or more other active ingredients or therapeutic agents, in addition to one or more peptoids disclosed herein.

One or more peptoids disclosed herein can also be administered in combination with other classes of compounds, including, but not limited to, agents to neutralize the anticoagulant activity of heparin, such as protamine; sepsis treatments, such as drotrecogin-α; antibacterial agents, such as ampicillin; antifungal agents such as terbinafine; anticoagulants, such as bivalirudin; thrombolytics, such as streptokinase; non-steroidal anti-inflammatory agents, such as aspirin; antiplatelet agents, such as clopidogrel; norepinephrine reuptake inhibitors (NRIs) such as atomoxetine; dopamine reuptake inhibitors (DARIs), such as methylphenidate; serotonin-norepinephrine reuptake inhibitors (SNRIs), such as milnacipran; sedatives, such as diazepham; norepinephrine-dopamine reuptake inhibitor (NDRIs), such as bupropion; serotonin-norepinephrine-dopamine-reuptake-inhibitors (SNDRIs), such as venlafaxine; monoamine oxidase inhibitors, such as selegiline; hypothalamic phospholipids; endothelin converting enzyme (ECE) inhibitors, such as phosphoramidon; opioids, such as tramadol; thromboxane receptor antagonists, such as ifetroban; potassium channel openers; thrombin inhibitors, such as hirudin; growth factor inhibitors, such as modulators of PDGF activity; platelet activating factor (PAF) antagonists; anti-platelet agents, such as GPIIb/IIIa blockers (e.g., abdximab, eptifibatide, and tirofiban), P2Y(AC) antagonists (e.g., clopidogrel, ticlopidine and CS-747), and aspirin; anti-coagulants, such as warfarin; low molecular weight heparins, such as enoxaparin; Factor VIIa Inhibitors and Factor Xa Inhibitors; renin inhibitors; neutral endopeptidase (NEP) inhibitors; vasopepsidase inhibitors (dual NEP-ACE inhibitors), such as omapatrilat and gemopatrilat; HMG CoA reductase inhibitors, such as pravastatin, lovastatin, atorvastatin, simvastatin, NK-104 (also known as itavastatin, nisvastatin, or nisbastatin), and ZD-4522 (also known as rosuvastatin, or atavastatin or visastatin); squalene synthetase inhibitors; fibrates; bile acid sequestrants, such as questran; niacin; anti-atherosclerotic agents, such as ACAT inhibitors; MTP Inhibitors; calcium channel blockers, such as amlodipine besylate; potassium channel activators; alpha-adrenergic agents; diuretics, such as chlorothlazide, hydrochlorothiazide, flumethiazide, hydroflumethiazide, bendroflumethiazide, methylchlorothiazide, trichloromethiazide, polythiazide, benzothlazide, ethacrynic acid, tricrynafen, chlorthalidone, furosenilde, musolimine, bumetanide, triamterene, amiloride, and spironolactone; thrombolytic agents, such as tissue plasminogen activator (tPA), recombinant tPA, streptokinase, urokinase, prourokinase, and anisoylated plasminogen streptokinase activator complex (APSAC); anti-diabetic agents, such as biguanides (e.g. metformin), glucosidase inhibitors (e.g., acarbose), insulins, meglitinides (e.g., repaglinide), sulfonylureas (e.g., glimepiride, glyburide, and glipizide), thiozolidinediones (e.g. troglitazone, rosiglitazone and pioglitazone), and PPAR-gamma agonists; mineralocorticoid receptor antagonists, such as spironolactone and eplerenone; growth hormone secretagogues; aP2 inhibitors; phosphodiesterase inhibitors, such as PDE III inhibitors (e.g., cilostazol) and PDE V inhibitors (e.g., sildenafil, tadalafil, vardenafil); protein tyrosine kinase inhibitors; antiinflammatories; antiproliferatives, such as methotrexate, FK506 (tacrolimus, Prograf), mycophenolate mofetil; chemotherapeutic agents; immunosuppressants; anticancer agents and cytotoxic agents (e.g., alkylating agents, such as nitrogen mustards, alkyl sulfonates, nitrosoureas, ethylenimines, and triazenes); antimetabolites, such as folate antagonists, purine analogues, and pyrridine analogues; antibiotics, such as anthracyclines, bleomycins, mitomycin, dactinomycin, and plicamycin; enzymes, such as L-asparaginase; farnesyl-protein transferase inhibitors; hormonal agents, such as glucocorticoids (e.g., cortisone), estrogens/antiestrogens, androgens/antiandrogens, progestins, and luteinizing hormone-releasing hormone anatagonists, and octreotide acetate; microtubule-disruptor agents, such as ecteinascidins; microtubule-stablizing agents, such as pacitaxel, docetaxel, and epothilones A-F; plant-derived products, such as vinca alkaloids, epipodophyllotoxins, and taxanes; and topoisomerase inhibitors; prenyl-protein transferase inhibitors; and cyclosporins; steroids, such as prednisone and dexamethasone; cytotoxic drugs, such as azathiprine and cyclophosphamide; TNF-alpha inhibitors, such as tenidap; anti-TNF antibodies or soluble TNF receptor, such as etanercept, rapamycin, and leflunimide; and cyclooxygenase-2 (COX-2) inhibitors, such as celecoxib and rofecoxib; and miscellaneous agents such as, hydroxyurea, procarbazine, mitotane, hexamethylmelamine, gold compounds, platinum coordination complexes, such as cisplatin, satraplatin, and carboplatin.

The disclosure further provides a method of manufacturing one or more peptoids disclosed herein comprising utilizing a submonomer process. In a particular embodiment, a method of manufacturing the one or more peptoids disclosed herein comprises the steps of:

(A) a resin bound amine or monomer, is coupled to bromoacetic acid using a peptide coupling agent, such as DIC, to form an attached submonomer potion;

(B) a second amine is then added to the submonomer portion by using a SN² reaction mechanism so as to form a full monomer,

wherein the amine nucleophile forms a bond with the electrophilic alkyl bromide carbon so that the bromine is displaced as a leaving group, and

wherein steps (A) and (B) are repeated until the peptoid is completely synthesized.

In another embodiment, a cleavage solution, such as 95:2.5:2.5 TFA/H₂O/TIPS, is added after the synthesis of the peptoid to cleave the peptoid from the resin and to remove any amine protecting groups. In yet another embodiment, the cleaved and synthesized peptoid is purified to remove any impurities, such as by using preparatory reverse phase HPLC.

In a further embodiment, the method of manufacture utilizes a peptide synthesizer which may or may not be further connected to a computer.

The disclosure also provides for use in the therapeutic applications described herein, kits and articles of manufacture are also described herein. Such kits can comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers can be formed from a variety of materials such as glass or plastic.

For example, the container(s) can comprise one or more peptoids described herein, optionally in a composition or in combination with another agent as disclosed herein. The container(s) optionally have a sterile access port (for example the container can be an intravenous solution bag or a vial having a stopper pierceable by a hypodermic injection needle). Such kits optionally comprise a compound with an identifying description or label or instructions relating to its use in the methods described herein.

A kit will typically comprise one or more additional containers, each with one or more of various materials (such as reagents, optionally in concentrated form, and/or devices) desirable from a commercial and user standpoint for use of a compound described herein. Non-limiting examples of such materials include, but are not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein. These other therapeutic agents may be used, for example, in the amounts indicated in the Physicians' Desk Reference (PDR) or as otherwise determined by one of ordinary skill in the art.

The following examples are intended to illustrate but not limit the disclosure. While they are typical of those that might be used, other procedures known to those skilled in the art may alternatively be used.

### EXAMPLES

Materials.

Triisopropylsilane (99%), piperidine (99.5%), (R)-(+)-methylbenzylamine (98%) (Rpe), (S)-(−)-methylbenzylamine (98%) (Spe), N-butylamine, N-propylamine, N-ethylamine, diisopropylethylamine, dimethylsulfoxide (DMSO), dimethylformamide (DMF) and methanol, methylamine (40% aqueous solution) were purchased from Sigma-Aldrich. N,N′-diisopropylcarbodiimide (DIC), and bromoacetic acid (97%) were obtained from TCI America. N-(tert-butoxycarbonyl)-1,4-diaminobutane, N-(tert-butoxycarbonyl)-1,5-diaminopentane, Trifluoroacetic acid (TFA), and N-(tert-butoxycarbonyl)-1,3-diaminopropane were obtained from Chem-Impex International Inc. Rink amide 100-200 mesh (MBHA) resin 0.56 mmole/g substitution was obtained from Novabiochem. N-methyl-2-pyrrolidone (NMP) was purchased from Alfa Aesar Inc. 1-H-pyrozole carboxamidine HCl was purchased from AK Scientific Inc. Coatest® Assay materials were obtained from Diapharma in the form of a Chromogenix Coatest® Heparin kit. Pooled human plasma was obtained from Precision BioLogic.

Solid State Peptoid Synthesis.

All peptoids were synthesized on Rink Amide MBHA resin on an Applied Biosystems 433A peptide synthesizer linked with a Macintosh PowerMac G4 876 MHz computer running Applied Biosystems SynthAssist software ver. 2.0. The software was modified so as facilitate peptoid synthesis by the submonomer method developed by Zuckermann et al. (J. Am. Chem. Soc. 1992, 114:10646-10647)

Synthesis of Peptoids of Disclosure.

The peptoids were synthesized using 200 mg of Rink amide resin per run. Solutions of the various amines were used at a 1M concentration in DMF. This concentration provided a high level of monomer formation. Up to 20% DMSO was added to the DMF solutions of 4 and 5 carbon chain-bearing amines to aid solubility and avoid clogging of the needle assembly of the synthesizer. The amine solutions (6 mL) were placed in synthesizer cartridges and sealed with septum caps in preparation for synthesis. All reagents and solvents were filled in bottles to sufficient volume and N₂ gas was provided for a successful run. The Chemistry software was loaded into the synthesizer from the computer. Synthesis of the complete peptoids varied from one to two days depending on length of peptoid sequence. The acylation reaction was complete in about 2 hours and the SN² reaction was complete in about 1 hour.

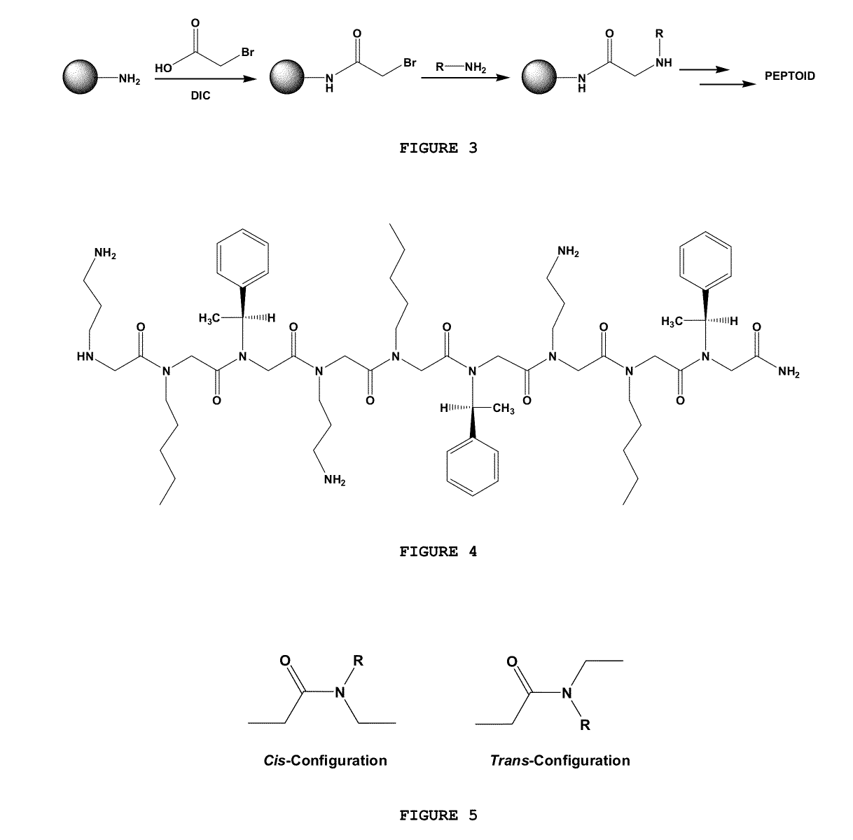

The generalized synthesis scheme for making the peptoids of the disclosure using the submonomer process is presented in FIG. 3. In the first step, a resin bound amine is coupled to bromoacetic acid using DIC to form an attached submonomer potion. A second amine is then added to the submonomer portion by a SN² reaction mechanism so as to form a full monomer, wherein the amine nucleophile forms a bond with the electrophilic alkyl bromide carbon so that the bromine is displaced as a leaving group. The process was continued until the peptoid was completely synthesized.

Following synthesis, the resin was first rinsed with methanol in vacuo, then a 95:2.5:2.5 TFA/H₂O/TIPS cleavage solution was added. After stirring the solution at ambient temperature for about 2.5 hours the resulting peptoid was cleaved from the resin and any amine protecting groups were also removed. The cleavage solution was rinsed from the funnel with methanol. The solvents were removed in vacuo to obtain the crude peptoid product as a semi-viscous yellow oil. The crude product was reconstituted with deionized water (5 mL) and then purified using preparatory reverse phase HPLC. The HPLC based solvents were removed in vacuo. The resulting purified peptoid product was taken up in deionized water (1 mL) and frozen using dry ice (Yield: 30-60 mg of peptoid per run). All peptoids were confirmed by using Matrix-assisted laser desorption/ionization time of flight (Maldi TOF) mass spectrometry. The peptoids that were produced by this method are provided in Table 2.

TABLE 2
Peptoid Designations and Sequences^{a}.

| | | | Molecular Weight (D) | |
|---|---|---|---|---|
| Peptoid Designation | | Sequence | Calculated | Found |
| 1. | s3mer | H-[N(Orn)-N(Bu)-N(Spe)]-NH2 | 407 | 406 |
| 2. | s3merG | H-[N(Arg)-N(Bu)-N(Spe)]-NH₂ | 448 | 471^{b} |
| 3. | s6mer | H-[N(Orn)-N(Bu)-N(Spe)]₂-NH₂ | 794 | 794 |
| 4. | s6merG | H-[N(Arg)-N(Bu)-N(Spe)]₂-NH₂ | 878 | 878 |
| 5. | s9mer | H-[N(Orn)-N(Bu)-N(Spe)]₃-NH₂ | 1183 | 1183 |
| 6. | s9merG | H-[N(Arg)-N(Bu)-N(Spe)]₃-NH₂ | 1309 | 1309 |
| 7. | s9mer(Pr) | H-[N(Orn)-N(Pr)-N(Spe)]₃-NH₂ | 1140 | 1141 |
| 8. | s9mer(Pr)G | H-[N(Arg)-N(Pr)-N(Spe)]₃-NH₂ | 1266 | 1267 |
| 9. | s9mer(Et) | H-[N(Orn)-N(Et)-N(Spe)]₃-NH₂ | 1098 | 1098 |
| 10. | s9mer(Et)G | H-[N(Arg)-N(Et)-N(Spe)]₃-NH₂ | 1225 | 1225 |
| 11. | s9mer(Me) | H-[N(Orn)-N(Me)-N(Spe)]₃-NH₂ | 1056 | 1079^{b} |
| 12. | s9mer(Lys) | H-[N(Lys)-N(Bu)-N(Spe)]₃-NH₂ | 1225 | 1225 |
| 13. | s9mer(Lys)G | H-[N(Lys)G-N(Bu)-N(Spe)]₃-N₂ | 1351 | 1351 |
| 14. | s9mer(Cad) | H-[N(Cad)-N(Bu)-N(Spe)]₃-NH₂ | 1267 | 1267 |
| 15. | s9mer(Cad)G | H-[N(Cad)G-N(Bu)-N(Spe)]₃-NH₂ | 1393 | 1394 |
| 16. | s9mer(All) | H-[N(Orn)-N(All)-N(Spe)]₃-NH₂ | 1134 | 1136 |
| 17. | s9mer(Bz) | H-[N(Orn)-N(Bz)-N(Spe)]₃-NH₂ | 1285 | 1285 |
| 18. | s12mer | H-[N(Orn)-N(Bu)-N(Spe)]₄-NH₂ | 1571 | 1571 |
| 19. | r12mer | H-[N(Orn)-N(Bu)-N(Rpe)]₄-NH₂ | 1571 | 1570 |
| 20. | s12merG | H-[N(Orn)-N(Bu)-N(Spe)]₄-NH₂ | 1739 | 1740 |
| 21. | s12merOrnOrn | H-[N(Orn)-N(Orn)-N(Spe)]₄-NH₂ | 1575 | 1575 |
| 22. | s12merArgArg | H-[N(Arg)-N(Arg)-N(Spe)]₄-NH₂ | | |

^{a}All peptoids confirmed by Maldi TOF following purification by HPLC.
^{b}M + Na

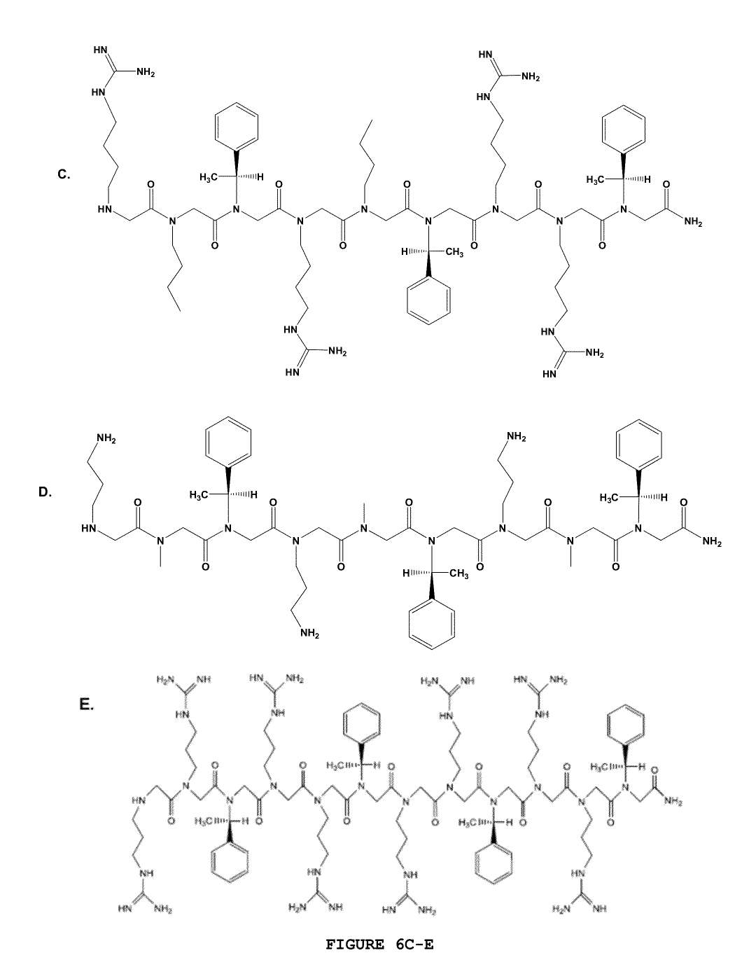

Structures for several of the peptoids disclosed herein are presented in FIG. 4 and FIG. 6. As can be seen in the figures, a variety of side chain substitutions were synthesized and many more can be envisioned using the process disclosed herein. Following synthesis and purification by reverse phase HPLC, peptoids of the disclosure were analyzed by circular dichroism (“CD”).

Circular Dichroism.

Information about the secondary structures of the peptoids was obtained by circular dichroism spectroscopy. CD spectra were measured on a Jasco J-815 CD spectrometer attached to a Dell Celeron D P.C. Sample solutions were contained in 1 mm quartz crystal QX cuvettes purchased from Fisher Scientific Inc. Nitrogen gas was used to purge the instrument for 5 minutes prior to and during the operation of the instrument to protect the optics and mirrors of the spectrometer.

CD Spectroscopy of Peptoids of Disclosure.

In a typical experiment, peptoids (200 μL) were diluted in deionized water to a volume (2 mL). The diluted peptoid (300 μL) was placed in the 1 mm cuvette and the CD spectrum was acquired using the following parameters: sensitivity=100 mdeg; wavelength range was 240 nm-190 nm; data pitch=1 nm, scanning speed=100 nm/min; scanning mode=continuous; response time=1 sec; accumulations=3. N₂ was used to purge the instrument at 20 psi for 5 min. before and throughout the operation of the spectrometer.

The first monomer in the trimer sequence of the peptoids disclosed herein comprised an alpha chiral side chain, the purpose of which was to produce a helical secondary structure. The placement of alpha chiral side chains in the first and in other positions in the sequence would predictably produce peptoids with polyproline type I helical structures. The chirality of the side chain was predictive of the handedness of the helix. Spe and Rpe are commonly used in this capacity. The S chiral monomer produces a right handed peptoid.

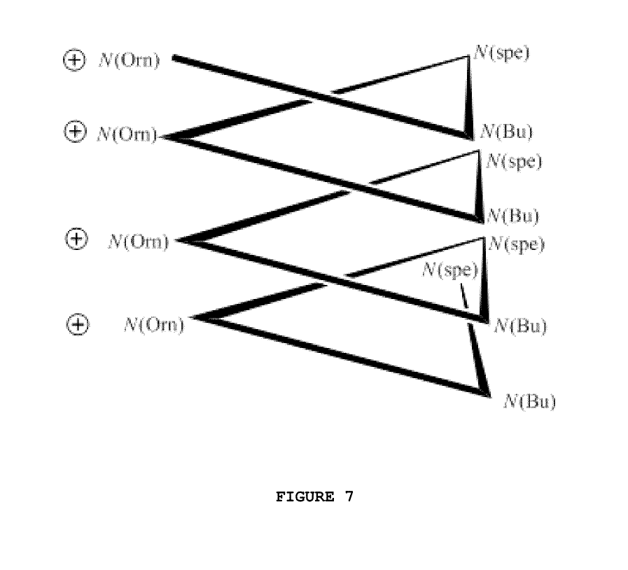

In the rational design of heparin binding peptoids disclosed herein, it was decided to place all cationic side chains so as that they would be located on one face of the peptoids polyproline type I helical structure (e.g., see FIG. 7). Since heparin consists of highly anionic sulfate esters and amides that are deprotonated at physiological pH, peptoids with cationic side chains should bind with high affinity to the anionic esters and amides of heparin.

Helicity of the peptoids disclosed herein were demonstrated by CD spectroscopy. The peptoids that were tested were built using a general scaffold consisting of the following sequence; H—[N(Cationic side chain)-N(alkyl or benzyl side chain)-N(spe)]₃-NH₂.

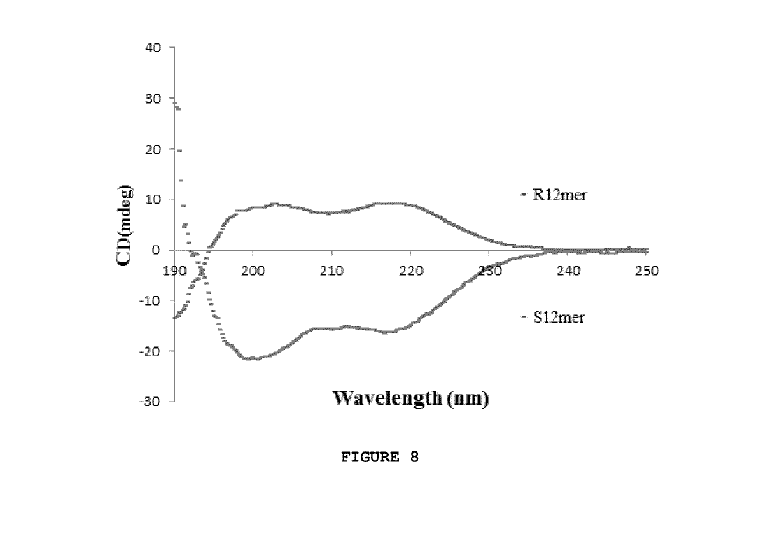

Two 12-mer peptoids (S12mer and R12mer in Table 3) were produced using the R and S forms of 1-phenylethylamine (Rpe and Spe) to determine the effect of chirality of the peptoid on the relative affinity of heparin binding. The resulting spectra are shown in FIG. 8. The S chiral peptoid produced spectra with double minima, shown in blue, which is consistent with a right handed helix. The R form produced the double maximum shown in red (see FIG. 8). Moreover, as seen in Table 3, ITC and HAC results indicate a greater binding affinity for the S form (Spe) to heparin than the R form.

| | | | ΔH | ΔS | ΔG |
|---|---|---|---|---|---|
| Peptoid | N | Kd(uM) | (cal/mole) | (cal/mole ° C.) | (cal/mole) |
| S12mer | 19.48 ± | 4.11 +/− | 1733 ± | 30.53 ± | −6309 |
| | 2.68 | 0.6 | 46.96 | 0.31 | |
| R12mer | 16.26 ± | 7.57 +/− | 1278 ± | 19.01 ± | −6956 |
| | 0.9 | 4.24 | 379 | 2.46 | |

For Table 3, binding constants and other thermodynamic parameters were determined for the chiral 12-mer analogs by ITC. The S12mer data represents 3 trials, and the R12mer represents 2 trials. Values for the parameters K_{b}, ΔH and N are obtained by fitting the titration curves. AG is calculated from ΔG=−RTlnK_{b} and ΔS is calculated from ΔG=ΔH−ΔS.

Regarding preference for right or left handedness and heparin binding affinity, the ITC data for the same 12-mers given in Table 3, indicate that either direction of the helix produces peptoids that bind; however, the S analogs possessed a higher affinity than the R-mer. Based upon the higher affinity of the S form, all other peptoids synthesized and studied in this research used the Spe chiral side chain.

Heparin Affinity Chromatography.

The relative affinities of the peptoids in Table 2 for heparin were determined by heparin affinity chromatography (“HAC”). Peptoid solutions, typically 0.2 mM, were eluted by gradient elution; mobile phase A consisted of pH 7.2, 50 mM sodium phosphate buffer and mobile phase B consisted of the same buffer but with 1 M NaCl added. The flow rate was 0.65 mL/min and the gradient began with 100% mobile phase A and 0% mobile phase B, and ended at 60 min with 0% mobile phase A and 100% B although most of the peptoid runs were complete within 30 min or less.

Following the fundamental choice of the chiral side chain to be used in the design and synthesis of the peptoids in this research, the next design variable to be explored was the effect of length of peptoids, i.e. the number of trimer repeats, and therefore the number of binding sites per peptoid on heparin binding affinity. To investigate the effect of length, and thus the number of cationic side chain binding sites, on heparin binding affinity, the binding of the series of peptoids H—[N(Orn)-N(Bu)-N(spe)]ₙ-NH₂, where n=1−4, was studied. In this series, the length of the peptoids vary from three to twelve monomer units and the number of ammonium groups vary from one to four.

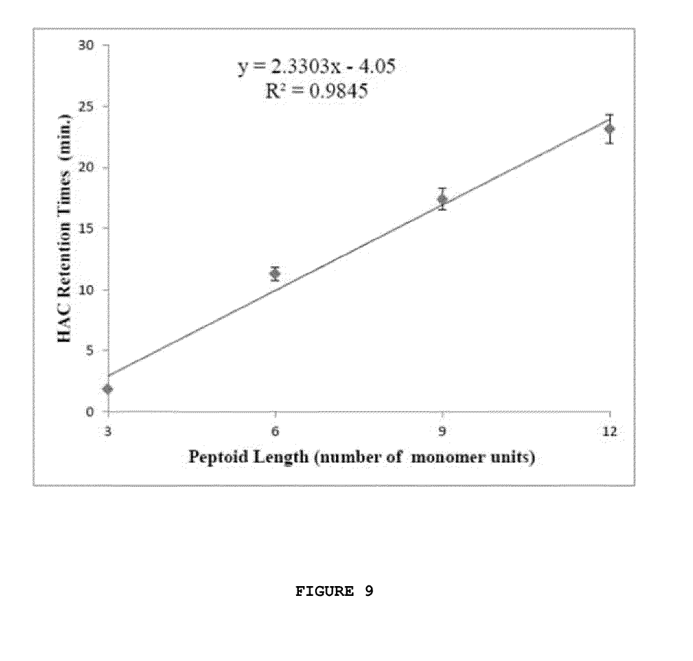

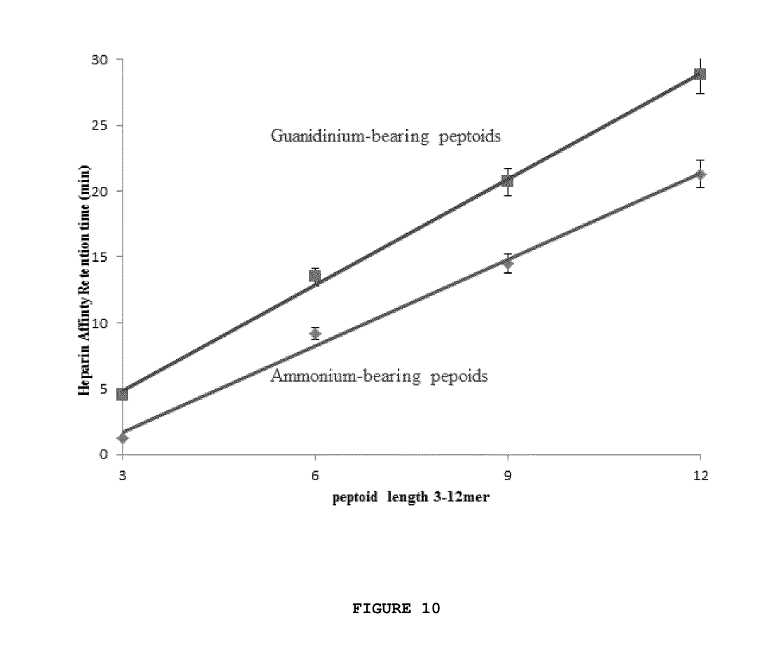

The HAC results for peptoids of length 3 to 12 are shown in FIG. 9 and FIG. 10. As expected there was a direct correlation between overall peptoid length and heparin binding affinity. However, peptoids longer than 12 units were found to excessively aggregate during ITC studies and so were not subsequently studied.

Binding Constants and Thermodynamic Parameters of Binding Reactions.

Isothermal Titration calorimetry (“ITC”) was used to determine directly ΔH, the binding constant and stoichiometry, and ΔS was then calculated from the binding constant and ΔH for the binding reaction between heparin and the peptoids produced in this study. In a typical ITC experiment, peptoid in buffer solution was placed in the sample cell and an equivalent quantity of the same buffer solution was placed in the reference cell. Heparin in buffer solution was placed in the titration syringe. Heparin titrant was added to the sample cell in 10 μL aliquots with 210 sec. between aliquots.

Length of Cationic Side Chain on Heparin Affinity.

The effect of the length of the carbon chain bearing the side chain ammonium and guanidinium groups, on heparin affinity was studied next. The carbon chains were varied from 3 to 5 carbons in length, corresponding to the amino acids ornithine, lysine and the diamine cadaverine. The side chain amine groups were also guanidinlyated to form the monomers N(Arg), N(Lys)G, and N(Cad)G, where N(Arg) is the N-substituted glycine analog of the amino acid arginine.

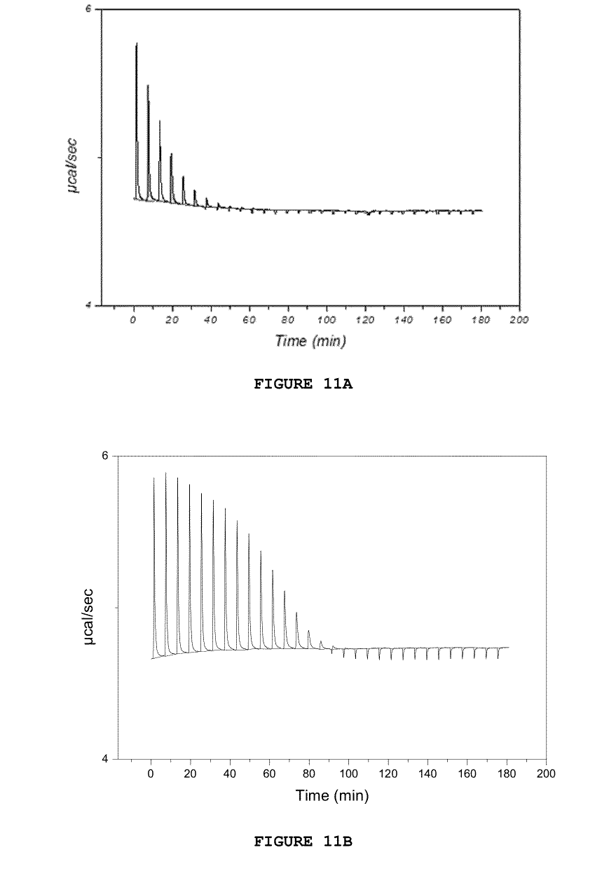

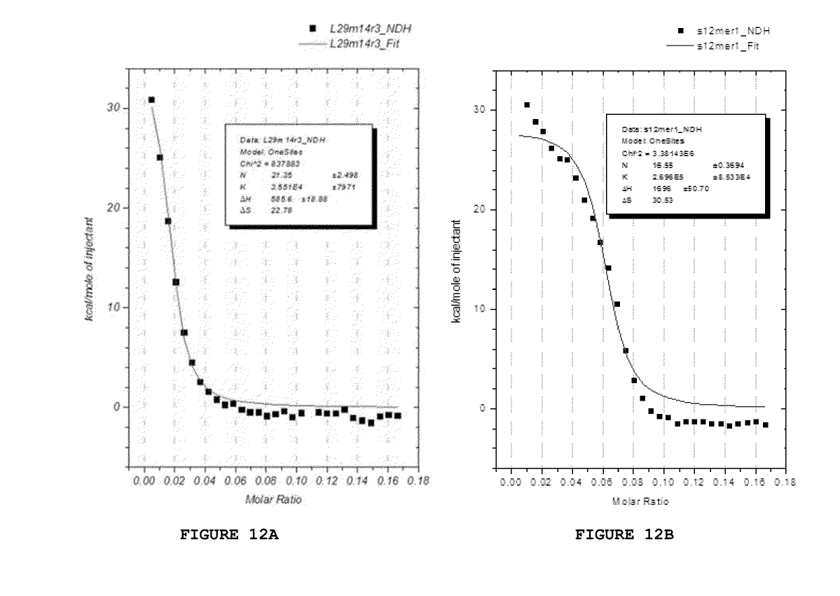

The ITC of a typical 9-mer is presented in FIG. 11 and FIG. 12. FIG. 11 presents the isotherm for the endothermic reaction. The resulting integrated titration curve is given in FIG. 12 for a reaction between 0.20 mM peptoid in the sample cell and 0.25 mM heparin in the titration syringe.

It is clear from the data for the primary amine-bearing side chains that extending the length of the carbon chain yields peptoids with greater heparin affinity as indicated by the binding constants. It is also apparent that, for the amine bearing peptoids, the reactions are endothermic in nature likely due to the displacement of sodium for the cationic ammonium groups electrostatically binding to the anionic sites on heparin.

The ITC data for guanidinylated peptoids provided less useful information as the Origin 5.0 software associated with the Microcal ITC was not able to resolve the raw data into a viable curve in order to provide the thermodynamic parameters, K values and stoichiometry normally obtained. This was due to an anomalous interaction such as intramolecular binding between multiple heparin molecules and one peptoid thereby resulting in aggregation.

Side Chain Modifications and Effect on Heparin Binding Affinity.

Since the general scaffold for the heparin binding peptoids consists of a repeating trimer sequence, with the first and subsequently following third positions of the peptoids consisting of Spe, thereby controlling the handedness of the resulting helical peptoids, this left two variable side chains amendable to modification. The central monomer was modified and found that substitutions in this position did, indeed effect heparin binding affinity. Modifications to this position consisted of straight and in one case a branched aliphatic hydrocarbon chain, allylic and benzylic side chains, the last two produced from the use of allyl amine and benzylamine.

ITC and HAC data indicated an inverse effect for the central alkyl side chain on heparin affinity; the shorter the alkyl side chain on the central nitrogen of the repeating trimer sequence the greater heparin affinity. This effect is most likely due to decreased steric hindrance between the shorter chains and the cationic side chain and the alpha chiral side chains. However an unwanted side effect is increased flexibility around the central amide bonds in the repeating trimer sequence causing cis/trans isomerism (Hamza, M., Dissertation, University of California at Riverside, 2010). A peptoid with a benzyl group in this position produced a peptoid with on order of magnitude greater affinity (Table 4), which could be due to enhanced rigidity yielding more backbone amide bonds in the trans conformation which would result in the ideal helical structure with more of the cationic side chains on the same face consistent with the rational design of the peptoids. Table 4 provides the thermodynamic parameters for Heparin binding peptoids as determined by ITC and HAC retention times.

TABLE 4
Thermodynamic parameters for Heparin binding peptoids
as determined by ITC and HAC retention times.

| | | | | ΔH | TΔS | HAC r.t. |
|---|---|---|---|---|---|---|
| No. | Peptoid | K_{d}(μM) | N | (cal/mole) | (cal/mole) | (min) |
| 1 | s3mer | 164 ± 32 | 25 ± 4.8 | 386.7 ± 237 | 5057 ± 35 | 3.0 ± 0.6 |
| 2 | s3merG | 140 ± 11 | 25 ± 2.1 | −632.5 ± 8.5 | 2026.4 ± 159 | 3.8 ± 0.3 |
| 3 | s6mer | 44 ± 5.0 | 18 ± 2.0 | 565 ± 64 | 7748 ± 880 | 9.61 ± 1.1 |
| 4 | s6merG | 20.6 ± 0.8 | 17 ± 0.7 | −2862 ± 222 | 2316 ± 32 | 13.5 ± 2.9 |
| 5 | s9mer | 18.6 ± 3.2 | 18 ± 2.7 | 814 ± 89 | 7259 ± 45 | 14.1 ± 1.2 |
| 6 | s9merG | 4.76 ± 2.3 | 20 ± 3.5 | −1197 ± 71 | 16.6 ± 1.7 | 20.8 ± 0.8 |
| 7 | s9merPr | 11.2 ± 0.7 | 17 ± 0.9 | 3555 ± 56 | 1427 ± 2.6 | 16.5 ± 0.7 |
| 8 | s9merPrG | 5.6 ± 1.8 | 24 ± 0.9 | −1634 ± 43 | 4639 ± 36 | 20.3 ± 0.6 |
| 9 | s9merEt | 10.9 ± 0.7 | 17 ± 0.9 | 822.3 ± 34 | 5140 ± 37 | 16.5 ± 0.5 |
| 10 | s9merEtG | 4.3 ± 2.2 | 20 ± 1.4 | −2176 ± 82 | 3159 ± 115 | 21.5 ± 0.5 |
| 11 | s9merMe | 8.3 ± 2.0 | 15 ± 0.4 | 1654 ± 11 | 3161 ± 76 | 18.1 ± 0.5 |
| 12 | s9merLys | 16.4 ± 11 | 16 ± 0.1 | 3305 ± 64 | 10,000 ± 54 | 14.6 ± 0.2 |
| 13 | s9merLysG | 5.0 ± 0.3 | 17 ± 3.4 | −615.1 ± 135 | 3948 ± 56 | 20.6 ± 0.2 |
| 14 | s9merCad | 11.7 ± 0.1 | 21.9 ± 0.3 | 2381 ± 29 | 8989 ± 15 | 16.3 ± 0.6 |
| 15 | s9merCadG | 5.5 ± 3.4 | 20 ± 1.4 | −283.5 ± 27 | 7500 ± 32 | 20.1 ± 0.5 |
| 16 | s9merAll | 8.3 ± 3.2 | 19.3 ± 2.4 | −931 ± 74 | 4627 ± 41 | 20 ± 2.0 |
| 17 | s9merBz | 0.9 ± 0.4 | 14 ± 0.2 | −545 ± 121 | 2851 ± 1.4 | 29.3 ± 0.5 |
| 18 | s12mer | 4.11 ± 0.6 | 19.5 ± 2.7 | 1733 ± 47 | 9097 ± 30 | 21.6 ± 0.6 |
| 19 | r12mer | 7.6 ± 4.2 | 16 ± 0.9 | 1278 ± 379 | 5664 ± 2.5 | 18.5 ± 0.6 |
| 20 | s12merG | 3.4 ± 0.4 | 19.7 ± 5.7 | −167 ± 38 | 7682 ± 43 | 29.2 ± 1.0 |
| 21 | s9merOrnOrn | 0.14 ± 0.004 | 8.7 ± 0.9 | −5253 ± 105 | 3992 ± 383 | 38.5 ± 5.0 |

For the cationic side chain, increasing the carbon chain from three to five carbons produced peptoids with highest affinity. This suggests that longer chains provide greater opportunity for the ammonium and guanidinium groups to reach the anionic binding sites of heparin.

In general, for ITC of this class of heparin binding peptoids, for peptoids with one ammonium bearing side chain, the enthalpy was endothermic, while in the case of two ammonium groups such as s12OrnOrn, or the guanidinium bearing peptoids, the enthalpy of reaction was exothermic.

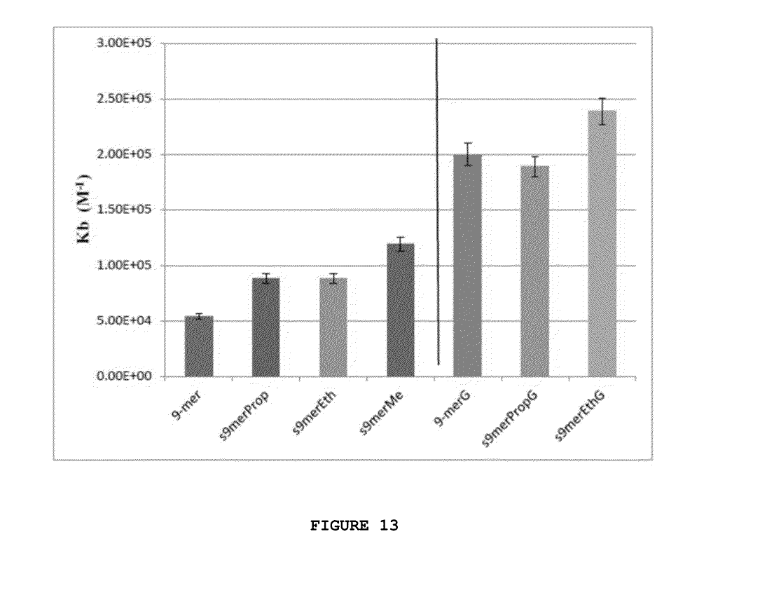

The results of HAC experiments were plotted as bar graphs and showed a significant effect of length of alkyl side chain on heparin affinity. The bar graph shown in FIG. 13 displays a pattern for peptoids both with ammonium and guanidinium bearing side chains. These chains are the third constituent monomer position and in both cases, the longer side chains result in higher binding affinity. Although in the case of peptoids having guanidinium bearing side chain, the guanidinium groups result in peptoids with equal affinity and the effect of the second or middle side chain is not as apparent. On the other hand, for ammonium bearing peptoids shown to the left side of the line in the chart, the effect is most apparent.

As FIG. 13 shows, the shorter the alkyl side chain on the middle residue of the repeating trimer sequence results in greater affinity for both ammonium bearing and guanidinium bearing peptoids, although the effect is overshadowed by the effect of guanidinylation but is still apparent.

Coatest Coagulation Assay.

Prior to running an assay, a calibration curve was constructed from 0.1 i.u./mL of heparin in the following concentrations: 0.01 i.u./mL, 0.03 i.u./mL, 0.05 i.u./mL and 0.07 i.u./mL of heparin in 500 μM Tris buffer pH 8.4. In addition to buffer and heparin solutions, plasma and AT were added to each calibration mixture. The method requires 200 μL of the standard solutions to be incubated for 3 min. at 37° C., followed by addition of 0.71 nkat of bovine factor Xa. The solution was then incubated for 30 sec. prior to addition of 0.2 μmole of chromogenic substrate S-2222. The solution was incubated for 3 min. and then quenched by adding 300 μL of 20% acetic acid. The solution was then transferred to a semimicro cuvette for measurement for absorption at 405 nm.

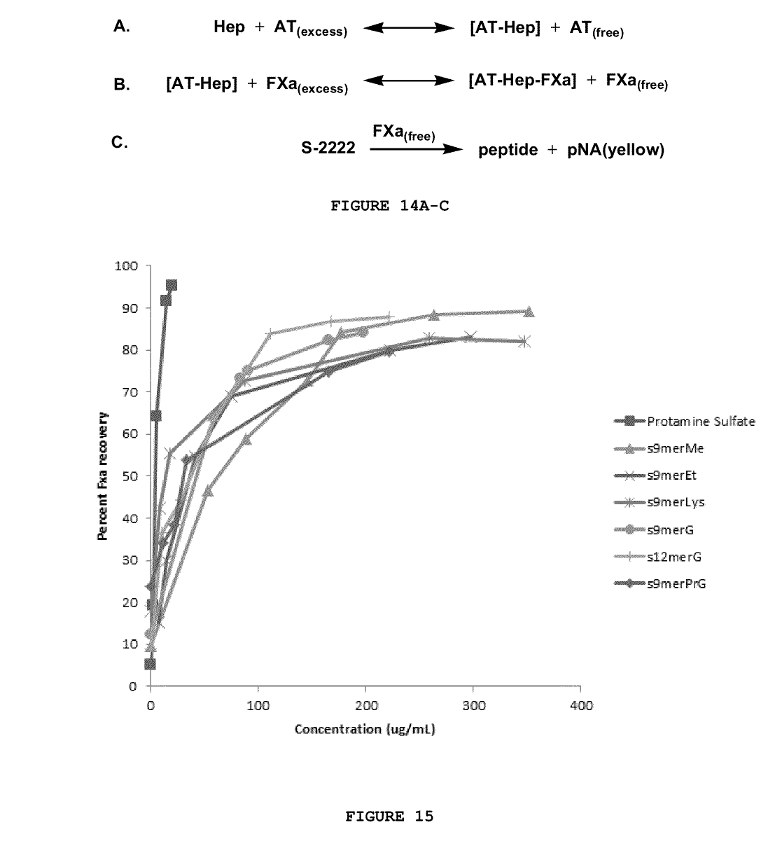

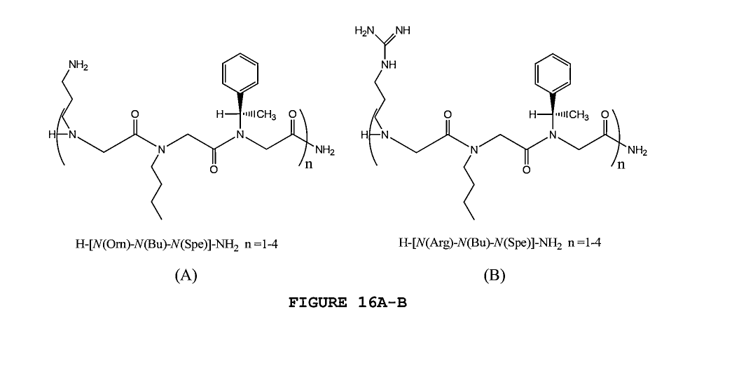

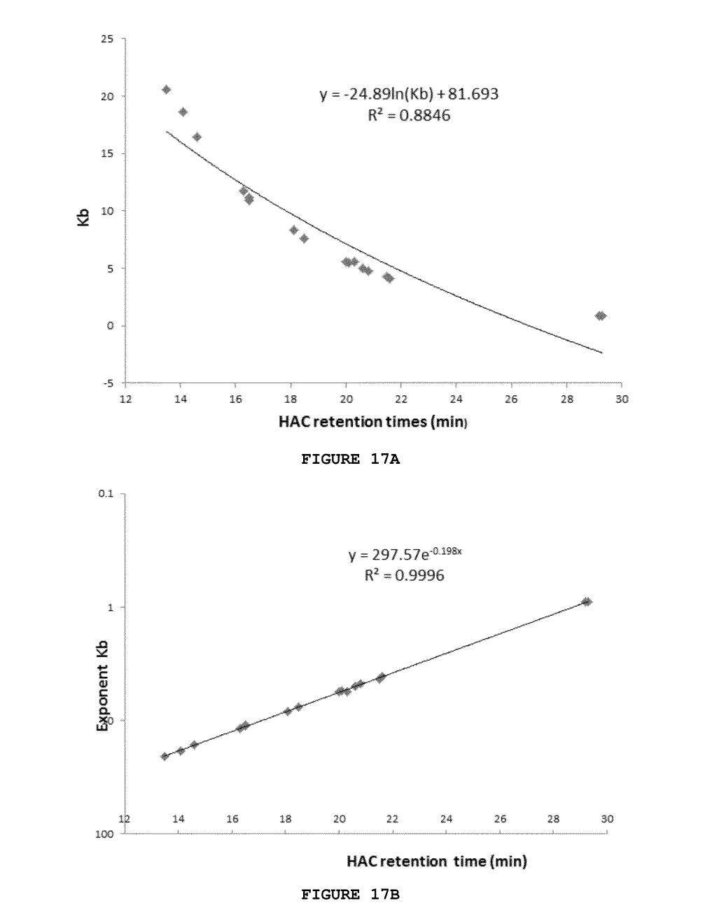

The above HAC, CD, and ITC methods provided useful binding affinity information and secondary structural data. The final assays conducted on the peptoids were done in order to study the anticoagulant activity of heparin while interacting with the peptoids produced. To gain this information the Coatest test was conducted on several of the peptoids produced in this study. The reaction schemes involved in the Coatest assay are chromogenic and the absorption at A₄₀₅ were measured from the chromogenic substrate S-2222, which consists of an aminopeptide Bz-ile-Glu(γ-OMe)-Gly-Arg bonded to p-NO₂-aniline, which produces a yellow color at 405 nm when cleaved by the excess FXa present in the reaction, the steps of which are shown in FIG. 14A-C.

Coatest Coagulation Assay for Peptoids of the Disclosure.

To produce the peptoid curves, concentrations of peptoids ranging from 0 to 2.5×10⁻⁴M were assayed in 0.07 i.u./mL heparin under the same conditions as above. The absorption values were converted to percent recovery and concentrations of peptoids were converted into micrograms per millimeter.

When peptoids are added with a high binding affinity for heparin, the [AT-Heparin-FXa] complex should be inhibited, resulting in greater quantities of FXa present, which in turn would cleave a proportionally larger quantity of chromophore and a higher absorption at 405 nm.

The Coatest assay was conducted on a variety of the peptoids produced in this study. A Coatest assay of six representative peptoids in comparison to protamine sulfate is presented in FIG. 15. The assay measures percent recovery of FXa required to restore heparin activity. As the concentration of peptoid is increased, the heparin is bound to the peptoid instead of the heparin-AT-FXa complex resulting in an increase in free FXa, which catalyzes the cleavage of the chromogenic substrate s-2222 shown in the reaction scheme given above (see e.g., FIG. 15).

A number of embodiments have been described herein. Nevertheless, it will be understood that various modifications may be made without departing from the spirit and scope of this disclosure. Accordingly, other embodiments are within the scope of the following claims.

What is claimed is:

1. A peptoid comprising the structure of Formula I(a):
wherein,
Z is an integer from 1 to 10;
R¹-R² are each individually
wherein R³ is (S)-1-phenylethylamine or (R)-1-phenylethylamine.

2. The peptoid of claim 1, wherein Z is either 3 or 4.

3. The peptoid of claim 1, wherein the peptoid forms a secondary structure similar to polyproline type I or polyproline type II peptide helices.

4. The peptoid of claim 3, wherein the peptoid forms a secondary structure similar to a polyproline type I helix where all of the R groups comprising cationic groups project from one face of the helix.

5. The peptoid of claim 1 having the structure

6. A pharmaceutical composition comprising a pharmaceutically acceptable carrier and a peptoid of claim 1.

7. A method of neutralizing the anticoagulant activity of heparin comprising administering a peptoid of claim 1.

8. The method of claim 7, wherein the peptoid is administered parenterally.

9. A method of neutralizing the anticoagulant activity of heparin comprising administering a pharmaceutical composition of claim 6.

10. The method of claim 9, wherein the pharmaceutical composition is administered parenterally.

11. A method of making a peptoid of claim 1 comprising:
(A) coupling a resin bound amine or monomer to bromoacetic acid using a peptide coupling agent to form an attached submonomer portion;
(B) adding a second amine to the submonomer portion using an SN² reaction mechanism so as to form a full monomer,
wherein an amine nucleophile forms a bond with an electrophilic alkyl bromide carbon such that the bromine is displaced as a leaving group, and
repeating (A) and (B) until the peptoid is synthesized.

12. The method of claim 11, wherein the peptide coupling agent is N,N′-diisopropylcarbodiimide.

13. The method of claim 11, wherein a cleavage solution is added after the synthesis of the peptoid to cleave the peptoid from the resin and to remove amine protecting groups.

14. The method of claim 13, wherein the peptoid is purified by using prepatory reverse phase HPLC.
